(19) United States
(12) Patent Application Publication
(10) Pub. No.: US 2006/0264926 A1
(43) Pub. Date: Nov. 23, 2006

(54) CUTANEOUS STABILIZATION BY VACUUM FOR DELIVERY OF MICRO-NEEDLE ARRAY

(76) Inventor: Gary S. Kochamba, La Canada, CA (US)

Correspondence Address: GREENBERG TRAURIG LLP, 2450 COLORADO AVENUE, SUITE 400E, SANTA MONICA, CA, 90404, US

(21) Appl. No.: 11/428,303

(22) Filed: Jun. 30, 2006

Related U.S. Application Data
(63) Continuation-in-part of application No. 10/906,602, filed on Feb. 25, 2005.
Application No. 10/906,602 is a division of application No. 10/290,819, filed on Nov. 8, 2002, now Pat. No. 6,896,666.

(51) Int. Cl. A61B 18/18

(52) U.S. Cl. 606/41; 604/93.01; 604/141

(57) ABSTRACT

A device permits effective engagement of micro-needles with a cutaneous layer thereby to permit for a substance to pass effectively to the cutaneous layer. A flexible or non-flexible material supports an array of micro-needles for receiving the surface of the cutaneous layer so that the proximal ends of the micro-needles pierce to effect a passage of a substance with the micro-needles to the cutaneous layer. A passageway or bladder acts on the substrate to cause the proximal ends of the micro-needles to pass to the cutaneous layer for passage of a substance associated with the micro-needles to the cutaneous layer. The bladder includes apertures located about and spaced from the micro-needles such that suction transmitted though the apertures. The micro-needles are mounted with a movable first substrate. In one form, the first substrate includes a surface with concavities and the micro-needles are mounted in the concavities, and the apertures are located in the concavities. In another form the surface is substantially flat. There can be a third layer spaced relative to the second substrate layer at least partly forming a chamber between the second layer and third layer. There can be a biasing device for urging the first substrate from the cutaneous layer.

### RELATED APPLICATION

This application is a continuation-in-part of U.S. application Ser. No. 10/906,602, filed Feb. 25, 2005, entitled CUTANEOUS INJECTION DELIVERY UNDER SUCTION, which is a divisional of U.S. application Ser. No. 10/290,819, filed Nov. 8, 2002 and issued as U.S. Pat. No. 6,896,666 on May 24, 2005, entitled CUTANEOUS INJECTION DELIVERY UNDER SUCTION. The contents thereof are incorporated by reference in their entireties.

### BACKGROUND OF THE DISCLOSURE

This disclosure relates to injection delivery cutaneously or subcutaneously. In particular, it relates to a system for stabilizing tissue then facilitating injection of fluids into a body which may be human or animal. More specifically it relates to micro-needles.

Many systems have been devised for the effective delivery of injectable material such as drugs into a body. These all suffer from one or another drawback.

A conventional method for administration is a hypodermic syringe, but this has disadvantages. Particularly, syringes may not be useful for self-administration by patients because of the dangers of embolisms arising from the introduction of air bubbles into the bloodstream, incorrect dosing and accidental infections.

Some syringes are pre-filled, which does correct some of the disadvantages, but difficulties, however, still arise with the complexity of manipulating the syringes in a smooth or uniform fashion with a single hand. Additionally, some patients have the fear of micro-needles and the sophistication and complexity of mechanical arrangements for activating self-administering syringes generally continues to be a disadvantage. Other disadvantages arise from the system for loading pre-prepared syringes with mechanisms and propellants to activate the syringes throughout the anticipated shelf life of the product.

Needle-less devices are known, but often these require superior dexterity for use and this is also a disadvantage for effecting injections by patients or doctors.

Many other disadvantages can arise from the complexity of different systems.

It is, accordingly, an object of the present disclosure to provide an injection delivery system which is capable of delivering preset dosages of a drug to a subject, is suitable for self-administration, does not require the conscious insertion of a needle into the skin, is simply constructed for mass production and, in different situations, can be repetitively used where there are mass dosages provided with the system.

The objects and advantages of the disclosure are set out further below.

### SUMMARY

According to the disclosure, there is provided a device, system and method of delivery of fluids by injection to the cutaneous or subcutaneous region of a living body under the effects of suction or a vacuum.

The device includes a housing having a peripheral edge and a needle in the housing for piercing the cutaneous layer. There is a bladder for containing fluid for injection below the cutaneous layer, and an area transversely within the peripheral edge of the housing and through which the proximal end of the needle may be directed. The area is for receiving the surface of the cutaneous layer about which the proximal end of the needle is to pierce to effect an injection of fluid. The area includes a surface, the surface being for receiving the cutaneous layer under action of the suction force. This stabilizes the cutaneous layer prior to and during piercing of the cutaneous layer by the proximal end of the needle.

There is a generator for generating a suction force at the area thereby to urge the cutaneous layer towards the area within the peripheral edge of the housing and thereby provide a stabilizing force to the cutaneous layer. A differential force such as a pressure, preferably suction, causes the needle to move through the transverse area and thereby pierce the surface of the cutaneous layer. The needle action can be under electromagnetic force or spring loaded.

The expulsion of fluid from the bladder into the distal end of the needle permits the expulsion of fluid through the proximal end of the needle.

The device includes a mounting for supporting a needle. The suction causes the needle and its mounting to move through the transverse area. The means for providing a suction force causes the movement of the needle mounting and thereby causes the needle to move between a position of repose relatively withdrawn from the transverse area and a position extending through the transverse area.

The device permits the bladder to move under a suction force towards the proximal end of the needle, and thereby permits the distal end to pierce the bladder and permits fluid from the bladder to enter the distal end of the needle and subsequently exit the proximal end of the needle. The bladder is formed in part of elastomeric material whereby the elastomeric material retains a force on the fluid in the bladder. This force can be applied directly or by a vacuum release switch. The fluid may be in a cartridge operated by pressure or spring or electromagnetic force.

Applying a further suction to the needle mounting permits movement of the plate a predetermined amount and thereby permits piercing of a sealed chamber in the housing. This causes venting of that suction force which causes the needle to be urged from the position of repose.

The device includes a biasing element means for causing the needle to be urged from the transverse area.

In one preferred form of the disclosure the transverse surface includes multiple ports through which suction can be applied to the surface. There can be multiple micro-needles in relative adjacency with each other thereby to permit multiple piercings of the cutaneous layer.

The device includes a suction generating chamber, and an inlet from the suction generating chamber into the housing for transmitting the suction to the housing. At least one secondary needle permits a pressure connection between the inlet for the suction and a ventilation chamber after a predetermined amount of movement of the needle whereby the suction force is vented to the ventilation chamber.

Venting of the suction force firstly permits the needle to be retracted from the exposed position, and thereafter permits the cutaneous layer to move from the transverse area.

In one preferred construction of the device, the housing is a cylindrical member with a circular outer edge. The transverse area is an inwardly concavely shaped area within the peripheral outer edge to permit the cutaneous surface to be drawn under suction to form a convex shape against the concave surface. The concave surface has multiple outlets surrounding a location for permitting passage of the needle through the area.

The ventilation chamber is removed from the transverse area. The needle mounting means is located between the transverse area and the bladder, which is located between the needle mounting means and the ventilation chamber.

In another aspect of the disclosure there is a signaling element or member for indicating the substantial completion of fluid expulsion from the needle. The signaling is selectively an audible signal, and the signal may be caused by the suction.

Preferably the needle is mounted with a movable plate, the plate having a biasing spring located between a block for holding the needle and the plate. The biasing acts to urge the block and needle from the plate, and the suction acts to urge the block towards the needle plate. The plate is mounted about its periphery with the internal wall of the housing. The mounting includes an elastic diaphragm thereby to permit movement of the plate under action of the biasing and the suction.

In yet a further construction of the device, the bladder is formed with a mounting plate for the needle. One wall of the bladder is the mounting plate, and there is a pierceable member with the mounting plate. Under suction, the plate is drawn towards the distal end of the needle, and the distal end of the needle is permitted to penetrate the pierceable member and enter the bladder. There is a normally sealed wall between the bladder and a ventilation chamber. Suction beyond a predetermined level causes the breakage of the sealed wall and thereby the venting of the suction from the transverse area.

The device of the disclosure preferably has the housing as an elongated structure. The needle is centrally located, and there is sequentially from a proximal end of the housing, firstly the transverse area including a surface through which the needle is adapted to move in an axial direction. Then there is a stabilizing block, one end of which forms the transverse surface. Ports are directed through the block from a side removed from the transverse surface. A guide block is provided for receiving a needle block so that the needle block is movable in the guide block. The guide block has ports to permit suction to pass to an axially movable needle mounting plate. The suction inlet to the housing is located between the guide block and the stabilizing block. The bladder is connected with the needle mounting plate, and the ventilation chamber is located on the opposite side of the bladder.

The device includes one or more secondary micro-needles to permit suction to pass from the suction inlet to the ventilation chamber when the needle plate is moved to a pre-selected position sufficiently close to the proximal end of the housing. The biasing means urges the needle plate to a position removed from the proximal end of the housing.

A method for delivery of a fluid cutaneously comprises generating a suction force on a surface of a housing thereby to receive under the suction force a surface of a cutaneous layer about which the proximal end of a needle is to pierce to effect an injection of fluid. A suction force operates the movement of a needle in the housing. The needle moves under the suction force from the housing thereby to permit piercing a cutaneous layer. The bladder for containing fluid is emptied into the distal end of the needle and thereby permits the expulsion of fluid through the proximal end of the needle for injection below the cutaneous layer.

More preferably the needle is moved through the transverse area under a suction force. This causes the needle to move between a position of repose relatively withdrawn from the transverse area and a position extending through the transverse area. The bladder is also moved under a suction force towards the distal end of the needle, and this permits the distal end to pierce the bladder and permits fluid from the bladder to enter the distal end of the needle and subsequently exit the proximal end of the needle.

In another aspect of the disclosure the method for delivery of a fluid subcutaneously comprises applying to the surface including multiple suction points. Multiple piercings are effected through multiple micro-needles in relative adjacency with each other. The bladder or drug cartridge moves under a suction force towards the proximal end of the micro-needles. This permits the distal end to pierce the bladder and permits fluid from the bladder to enter the distal end of the micro-needles and subsequently exit the proximal end of the respective micro-needles.

In another aspect of the disclosure, a device permits effective engagement of micro-needles with a cutaneous layer thereby to permit for a substance to pass effectively to the cutaneous layer. A flexible or non-flexible material supports an array of micro-needles for receiving the surface of the cutaneous layer so that the proximal ends of the micro-needles effect a passage of a substance with the micro-needles to the cutaneous layer. A passageway or bladder acts on the substrate to cause the proximal ends of the micro-needles to pass to the cutaneous layer for passage of the substance to the cutaneous layer.

The bladder or passage way includes apertures located about and spaced from the micro-needles such that suction transmitted though the apertures. A second layer, together with the first layer forms the bladder or passageway. The micro-needles are mounted with the movable first substrate.

In one form the first substrate includes a surface with concavities and the micro-needles are mounted in the concavities, and the apertures are also located in the concavities. In another form the surface is substantially flat.

There can be a third layer spaced relative to the second substrate layer at least partly forming a chamber between the second layer and the third layer. There can be a biasing device for urging the first and/or second substrate from a position relatively removed from the cutaneous layer.

The needle array is micro-needles typically measuring about 25 to about 300 microns in height. In some cases the needles can each be about 1 to 50 microns in cross section, and have a spacing of about 150 microns between the needles. Selectively the material is biodegradable, and selectively made of silicon, silicon dioxide, metals, polymers, or composites. The fabrication process includes a MEMS process. The micro-needles area formed with a density of about 400 or more micro-needles in a manner to create many hundreds of micro-needles per square centimeter.

In another form of the disclosure, the device permits effective engagement of needles with a cutaneous layer thereby to permit for energy to pass effectively to the cutaneous layer. There is a first substrate of flexible material having a transverse area and a peripheral edge; and an array of needles arranged about the area for effectively engaging the cutaneous layer. A bladder related to the substrate acts on the substrate for causing proximal ends of the needles to pass to the cutaneous layer for passage of energy associated with the needles to the cutaneous layer.

The area transversely within peripheral edge of the material and with which the needles are connected are for receiving the surface of the cutaneous layer with which the proximal ends of the needles are to engage to effect a passage of energy with the needles to the cutaneous layer.

There is a connector for a suction generator for creating a suction force in the bladder thereby to urge the cutaneous layer towards the area within the peripheral edge of the material.

The arrangement is such that suction causes the needles and cutaneous layer to move relatively closer to each other and thereby permit the proximal ends of the needles to engage the surface of the cutaneous layer, and permit the passage of energy from the needles to the cutaneous layer.

There is also a form of a device for penetrating a cutaneous layer to permit for interaction with the cutaneous layer. This includes a first substrate of material having an area and a peripheral edge, and an array of needles arranged about the area for interacting with the cutaneous layer, the needles having a proximal end and a distal end.

Selectively, a spring or electromagnetic system causes the needles to move relatively with the cutaneous layer. An area transversely within peripheral edge of the material and through which the needles are directed, is for receiving the surface of the cutaneous layer about which the micro-needles are to interact.

The cutaneous layer is urged towards the area within the peripheral edge of the material and thereby provides a stabilizing force to the cutaneous layer. The arrangement is such that the needles and cutaneous layer are caused to move relatively closer to each other and thereby permit the proximal ends of the needles to interact with the surface of the cutaneous layer. This permits the interaction of the distal end of the needle with the cutaneous layer.

There can, in some situations, be a source of energy, the energy being selectively one of cryoenergy, ultrasound energy, RF energy, or other electromagnetic energy. The different micro-needles can have different electrical polarities, and can be uni-polar or bi-polar.

In some uses applying energy attains increased porosity of the cutaneous layer and permits for the inflammation of the cutaneous layer. In other uses there is an ablation of cutaneous or subcutaneous material or cells. The micro-needles can interact with the cutaneous layer to enhance the porosity or permeability of the layer thereby permitting the enhanced absorption of substances. A delivery of a substance can attain increased porosity of the cutaneous layer permitting for the inflammation of the cutaneous layer.

In some uses applying electromagnetic energy through needles or micro-needles creates heat and at about 50 degrees Celsius causes irreversible cell death and tissue ablation. Needle delivery of energy increases the depth of ablation and uniformity. fluid irrigation with, for example, saline, through the hollow needle decreases the impedance at tissue contact. This prevents tissue sticking to the ablation needle and prevents tissue char.

### DRAWINGS

FIG. 1 is a sectional side view of a device according to the disclosure in a first state;

FIG. 2 is a sectional side view of the device in a second state;

FIG. 3 is a sectional side view of the device in a third state;

FIG. 4 is a sectional side view of the device in a fourth state;

FIG. 5 is a sectional side view of an alternative device;

FIG. 6 is a plan bottom view of a multiple needle device;

FIG. 7 is a side view of FIG. 6 without suction;

FIG. 8 is a side view of FIG. 6 with suction;

FIG. 9 is a side view of FIG. 6 during drug delivery;

FIG. 10 is a side view of the interface with an ablation device;

FIG. 11 is a side view of the interface connecting the drug port to a pump;

FIG. 12 is a side view of a needle with a sensor;

FIG. 13 is a side view of the system with porous material and with two suction ports;

FIG. 14 is a side view with the needle orientated at 90°;

FIG. 15 is a side view with the needle orientated at 45°;

FIG. 16 is a side view of micro-needles with electromagnetic mechanisms;

FIG. 17 is an under view of a flexible pad illustrating a micro-needle array and an array of holes for a suction or vacuum creating array;

FIG. 18 is a side view of a flexible array of the micro-needles;

FIG. 19 is a top view of the flexible array showing the passageways for the suction creating path;

FIGS. 20A and 20B are a side view of different alternative micro-needle arrays;

FIG. 21 is a side view of an alternative non-flexible patch using a micro-needle array.

FIG. 22 is a side view of a different micro-needle array of a delivery patch with an adhesive pad;

FIG. 23 is a side view of a different micro-needle array with a sensor to stop the motion and a release mechanism for permitting withdrawal of the patch.

FIG. 24 is a side view of a different micro-needle array of a delivery patch with an adhesive pad, the delivery being an energy in the form of ultrasound energy or cryoenergy, RF energy or other electromagnetic energy.

### DETAILED DESCRIPTION

The disclosure is described with reference to the accompanying drawings.

A device, system and method is for delivery of fluids by injection to the cutaneous or subcutaneous region of a living body under the effects of suction or a vacuum.

The device 20 includes a housing 22 having a peripheral edge 24 and a needle 26 in the housing for piercing the cutaneous layer 28. There is a bladder 30 for containing fluid 32 for injection below the cutaneous layer 28, and an area 34 transversely within peripheral edge 24 of the housing 22 and through which the proximal end 36 of the needle 26 may be directed.

The area 34 is for receiving the surface 38 of the cutaneous layer 28 about which the proximal end 36 of the needle 26 is to pierce to effect an injection of fluid 32. The area 34 includes a surface 40, the surface being for receiving the cutaneous layer 28 under action of the suction force, and thereby stabilize the cutaneous layer prior to and during piercing of the cutaneous layer by the proximal end 36 of the needle 26.

There is a generator device element or means 42 for generating a suction force at the area 34 thereby to urge the cutaneous layer 28 towards the area within the peripheral edge 24 of the housing 22 and thereby provide a stabilizing force to the cutaneous layer 28. The suction causes the needle 26 to move through the transverse area 34 and thereby pierce the surface 38 of the cutaneous layer 28.

The suction effect permits the expulsion of fluid 32 from the bladder 30 into the distal end 48 of the needle 26 and thereby permits the expulsion of fluid 32 through the proximal end 36 of the needle 26.

The device includes a mounting means, member support element or device 40 for supporting a needle, and the suction causes the needle to move through the transverse area 34 to cause the movement of the needle block 54 and thereby cause the needle 26 to move between a position of repose relatively withdrawn from the transverse area 34 and a position extending through the transverse area.

The device permits for and includes a means, element or device for permitting the bladder 30 to move under a suction force towards the proximal end 36 of the needle 26, and thereby permit the distal end 44 to pierce the bladder or an entry 48 to the bladder 30 and permit fluid from inside the bladder to enter the distal end of the needle and subsequently exit the proximal end of the needle.

The bladder 30 is formed in part of elastomeric material whereby the elastomeric material retains a force on the fluid 32 in the bladder 30.

Applying further suction to the needle mounting plate 74 permits movement of a plate 50 a predetermined amount and thereby permits piercing of a sealed chamber 52 in the housing 46. This causes venting of the suction force which causes the needle 26 and its cylindrical block 54 back to the position of repose.

The device includes a biasing spring 56 for causing the needle 26 to be urged from the transverse area 34. Biasing the needle 26 urges the needle 26 from the transverse area 34. The biasing effect is operable selectively after the needle 26 has been urged into the cutaneous region 28 for a predetermined distance. Further, the biasing action by the spring 56 is selectively effective after the bladder 30 has been substantially emptied.

The transverse surface 40 includes multiple ports 58 through which suction can be applied to the surface 40. There can be multiple micro-needles 26 in relative adjacency with each other thereby to permit multiple piercings of the cutaneous layer. This embodiment is shown in FIGS. 6 to 9. A suitable port 60 is provided to deliver suction to cause needle movement. Another port 62 is provided to deliver a drug to the needle 26.

The device includes the suction-generating chamber 42, an inlet 64 from the suction-generating chamber 42 into the housing 22 for transmitting the suction to the housing. There is at least one secondary needle or valve 66 for permitting a pressure connection between the inlet 38 for the suction and a ventilation chamber 68, after a predetermined amount of movement of the needle 26 whereby the suction force is vented to the ventilation chamber 68.

Venting of the suction force firstly permits the needle 26 to be retracted from the exposed position, and thereafter permits the cutaneous layer 38 to move from the transverse area 34.

The construction of the device 20 includes the housing 22 which is a cylindrical member with a circular outer edge 24. The transverse area 34 is an inwardly concavely shaped area within the peripheral outer edge 24 to permit the cutaneous surface 38 to be drawn under suction to form a convex shape against the concave surface. The concave surface has multiple outlets 58 surrounding a location 70 for permitting passage of the needle 26 through the area 34.

The ventilation chamber 68 is removed from the transverse area 34. There is the needle mounting means 46 between the transverse area 34 and the bladder 30 is located between the needle 26, mounting means 46, and the ventilation chamber 68.

There is a signaling device, element or whistle means 72, for indicating the substantial completion of fluid expulsion from the needle 26. The signaling is selectively an audible signal, the signal being caused by the suction.

The needle 26 is mounted with a movable plate 74 of the mounting means 46. The plate 74 has the biasing spring 56 located between block 54 for holding the needle 26 and the plate 74. The biasing acts to urge the block 54 and needle 26 from the plate 74, and the suction acts to urge the block 54 towards the needle 26 and plate 74. The plate 74 is mounted about its periphery with the internal wall 76 of the housing. The mounting includes an elastic diaphragm 78 thereby to permit movement of the plate 74 under action of the biasing and the suction.

The bladder 30 is formed with the mounting plate 74 for the needle 26. One wall of the bladder 30 is the mounting plate 74, and there is the pierceable member 48 of the mounting plate 74. Under suction, the plate 74 is drawn towards the distal end 44 of the needle 26, and the distal end 44 of the needle 26 is permitted to penetrate the pierceable member 48 and enter the interior of the bladder 30.

There is a normally sealed wall 50 between the bladder 30 and the ventilation chamber 68, and the suction beyond a predetermined level causes the breakage of the sealed wall 50 at closed ports 80 and thereby the venting of the suction from the transverse area 34.

The housing 22 is an elongated structure. The needle 26 is centrally located, and there is sequentially from a proximal end or edge 24 of the housing, firstly the transverse area 34 including a surface through which the needle 26 is adapted to move in an axial direction. Then there is a stabilizing block 82, one end of which forms the transverse surface 34. Ports are directed through the block from a side removed from the transverse surface 34.

A guide block 84 is provided for receiving a needle block 54 so that the needle block 54 is movable in the guide block 84. The block 84 has ports 86 to permit suction to pass to an axially movable needle mounting plate 74. The suction inlet 64 to the housing 22 is located between the guide block 84 and the stabilizing block 82. The flexible elastic part of the bladder 30 is connected with the needle mounting plate 74, and the ventilation chamber 86 is located on the opposite side of the bladder 30 and also the opposite side of the plate 50.

The device 20 may include one or more secondary micro-needles or valves 66 to permit suction to pass from the suction inlet to the ventilation chamber 68 when the needle plate 74 is moved to a pre-selected position sufficiently close to the proximal end of the housing 22. The biasing means 56 urges the needle plate 74 to a position removed from the proximal end of the housing.

The method for delivery of a fluid cutaneously comprises generating a suction force on a surface area 34, of a housing 22 thereby to receive under the suction force, the surface 38 of the cutaneous layer 28 about which the proximal end of a needle 26 is to pierce to effect an injection of fluid.

The generated suction force operates the movement of the needle 26 in the housing 22. The needle 26 is moved under the suction force from the housing thereby to permit piercing a cutaneous layer.

The bladder 30 for containing fluid is emptied into the distal end 44 of the needle 26 and thereby permits the expulsion of fluid through the proximal end 36 of the needle 26 for injection below the cutaneous layer.

The needle 26 is moved through the transverse area 34 under the suction force and thereby causes the needle 26 to move between a position of repose relatively withdrawn from the transverse area 34 and a position extending through the transverse area 34. Also, the bladder 30 is moved under the suction force towards the distal end 44 of the needle 26, and thereby permits the distal end 44 to pierce the bladder cavity and permit fluid from inside the bladder to enter the distal end 44 of the needle 26 and subsequently exit the proximal end 36 of the needle 26.

In another aspect of the method for delivery of a fluid cutaneously, the surface 34, including multiple suction ports 58 is applied to the surface 38. Thereafter, multiple piercings through multiple micro-needles 26 in relative adjacency with each other are applied. The bladder 30 moves under a suction force towards the proximal end of the micro-needles 26, and thereby permits the distal ends to pierce a wall of the bladder 30 and permit fluid from the bladder 30 to enter the distal ends of the micro-needles 26 and subsequently exit the proximal ends of the respective micro-needles 26.

Many variations of the disclosure are possible. There can be various and additional chambers to those described above. The suction or vacuum producing chamber 42, the ventilation chamber 68 and the fluid or drug bladder 30 do not necessarily need to be in axial relationship with each other in the order that appear in the preferred embodiment. The chambers may be located on the left and right sides of each other in the device.

The suction ports 58 may be on one side of the central axis of the device. This may, in some situations, cause an imbalanced operation of the suction, with one side of the device experiencing the suction before the other side of the device. Alternative embodiments could balance the suction by utilizing a plurality of suction ports as described, for example two ports, one on each side, or an annular tube either on the outside or the inside of a suction chamber. The tube can have apertures through which the suction could be delivered equally into all parts of the suction chamber. The diameter of each of the openings in the annular tube is optimized with larger openings on one side and smaller on the other to make sure the suction is equal on all sides.

Some advantages of the suction features of the disclosure include the following. The suction draws the tissue or cutaneous or subcutaneous regions 28 up towards the edge 24 of the housing 22. This stabilizes the tissue prior to entry by the needle 26 into the tissue. The tissue is pulled up and away from other structures that could be damaged by the needle such as bone, tendons and nerves. When the needle 26 enters, the tissue is already stable.

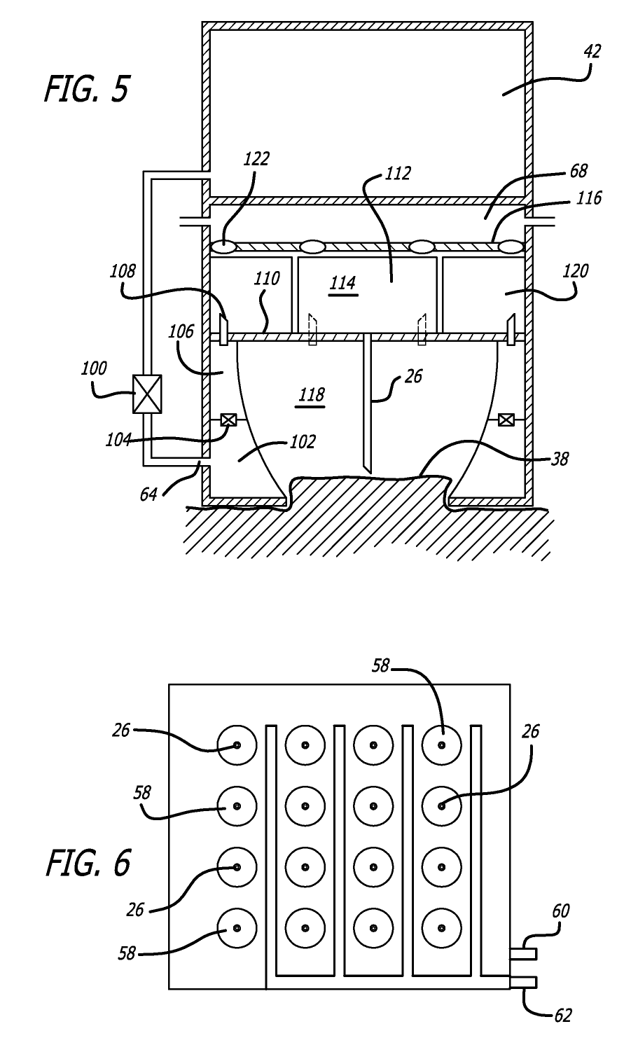

In FIG. 5 there is shown an embodiment where the suction generator 42 is located above the ventilation chamber 68. The suction chamber is connected through a conduit with a valve 100 to the inlet port 64. The port 64 goes into a bladder 102, which is circumferentially around the inner side of the housing. The bladder 102 is connected through a second valve 104 with a secondary circumferential bladder 106 and there is a needle 108 between a needle plate 110 on which a bladder 112 is mounted to contain fluid 114. A moveable drug plate 116 is located about the drug bladder 112 and forms the base of the ventilating chamber 68. The needle 26 is connected with the drug bladder.

The operation of the system is such that suction from the suction chamber 42 causes the bladder 102 to compress and then ultimately the bladder 106 to compress. As this happens, the needle 26 is drawn downwardly towards the cutaneous layer 38, which is drawn into a chamber-type formation 118. This causes interaction with the proximal end of the needle 26 as the needle plate 110 moves downwardly. The drug plate also moves downwardly and this causes the drug bladder to compress and release fluid 114 through the needle. At a predetermined point the needle 108 pierces the wall of the peripheral bladder 120 and then in turn pierces the drug plate 116 at the membranes 122. This causes a release or ventilation of the suction, which would otherwise cause the needle 26 to be drawn downwardly and outwardly. The needle 26 then retracts into the device.

In FIG. 6 there are shown multiple needles 26 which are arranged in a rectilinear array. There is the suction port 60 and suction port 62 located at one end of the relatively square profile of the housing for the device. As illustrated in FIG. 6 the system appears as a 16-cell system. The operation can be that each one of the micro-needles 26 operates sequentially as required. The system is useful for drug delivery and replacement of different plasma and blood components in angiogenesis and in cell transplant technology, namely myogenesis.

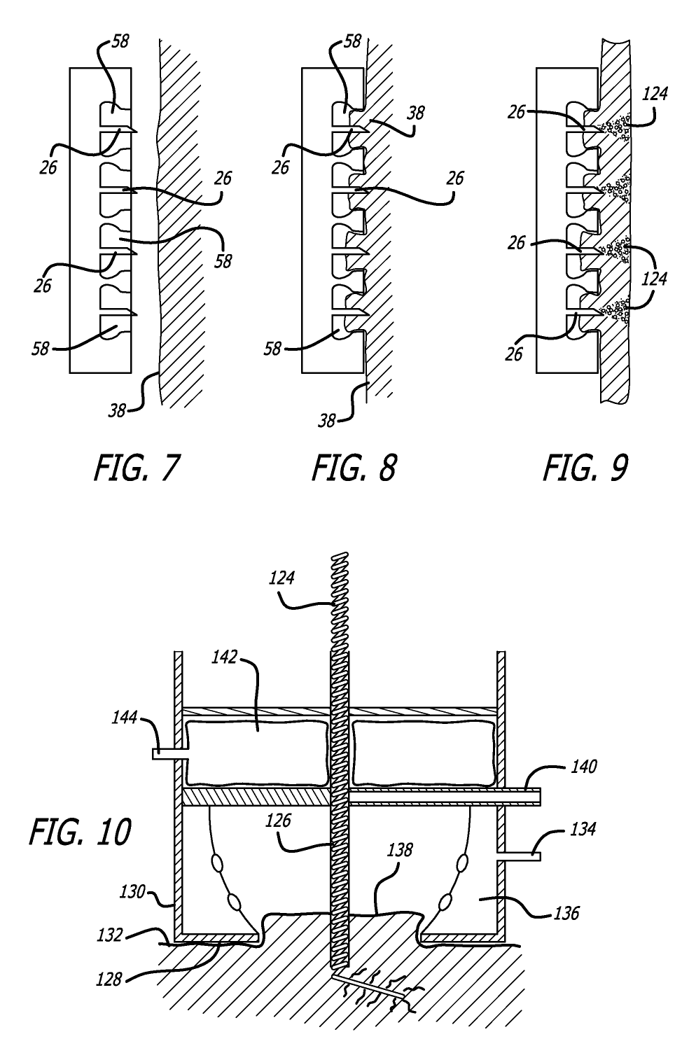

As shown in FIG. 7 there is a micro-needle array without suction and where the tissue interface 38 is slightly spaced from the tips of the needles. When suction is applied to the port 60, the tissue 38 is drawn towards the interface at the end of the device. The needle tips descend and penetrate the tissue as shown in FIG. 8. When suction or pressure is applied through port 62 the drug is delivered through the array into the tissue. This is illustrated in FIG. 9 with the drug being shown as drops 124. When the suction is removed the micro-needles retract and the array is removed.

In another form of the disclosure there can be configurations where there are peripheral bladders 120 around the drug bladder 114 which act to stabilize the device. This ensures the effective operation between the needle plate 110 and the drug plate 116.

In yet other forms of the disclosure there can be a configuration with spring members between the needle plate 110 and a structure below the needle plate towards the interface at the engaging end of the device. One or more of the spring members assist in the descent or recoil of the needle plate and the needle in the device. Alternatively MEMS may be used to deliver the needle plate and the needle.

As illustrated in FIG. 10 there is a configuration for use of the device with endocardial or intramycardial tissue. Such a device would operate in a linear or shaped array. It may be used to deliver fluids or energy therapy prior to drug delivery or in lieu of drug delivery. The therapy may include the removal of tissue scar or allow space for drug or cellular delivery. It could also include removal of tissue by true cut needle or delivery of an ablation needle. The ablation device may be a microwave, radio frequency (rf), laser or cryotherapy device. The energy delivered to tissue by the micro-needle can cause inflammation or ablation of living cells for medical therapy such as cosmetic regeneration, cancer therapy or cardiac rhythm control. The ablation device rf 124 as illustrated in FIG. 10 penetrates a bore 126 or is incorporated into a micro-needle or array. The proximal end 128 of the device 130 can be used for engagement of the tissue 132. When the appropriate suction is applied to port 134, the bladder 136 which is circumferentially located inside of the housing contracts appropriately and causes the tissue 132 to be sucked into the centralized portion of the aperture of the device and thereby forms an inset surface portion 138. Wherein stabilization of the tissue is achieved in this manner the ablation device can be inserted through the bore 126 or an ablation micro-needle array is delivered. Drugs can also be delivered through the port 140 which thereby enters the bore 126 as required. The bladder 142 surrounds the bore 126 and a suitable port 144 is activated to cause the bladders to inflate or deflate as required.

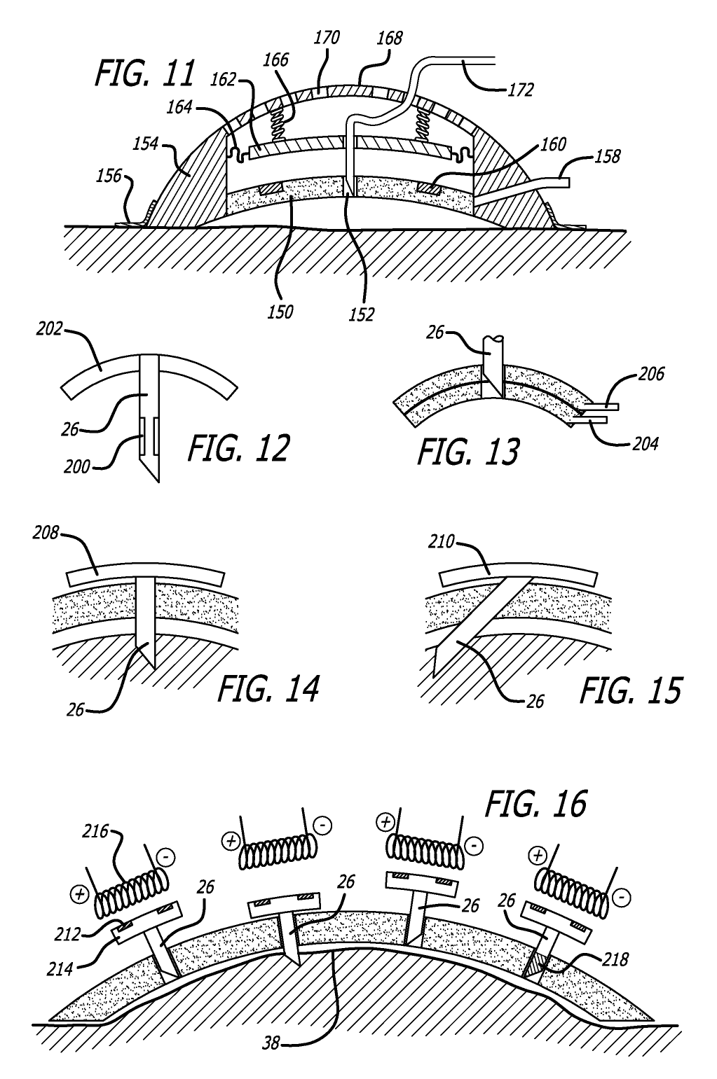

As illustrated in FIG. 11, there is shown a device that is used as a needle delivery system. This permits for automatic needle delivery without a self-sticking requirement. In some uses of the device as shown in the Figures, a peripheral self-stick arrangement may be provided circumferentially about the outer periphery at the interface 24 of the cutaneous material. Such material can assist in stabilization of the device against the cutaneous layer.

In FIG. 11 the needle penetrates skin during drug delivery. With this device and other devices of the disclosure, a smaller needle, such as a micro-needle, can be used and this causes less pain. It can be easily removed and replaced or relocated as necessary. The device can incorporate several needles and/or sensors. It can be connected to an i-pump with vacuum capability. In FIG. 11 there is a porous material 150 and a needle port 152 centrally located in the porous material. The porous material is mounted inside a housing 154 which can be secured with tape or fastener 156 to the tissue. The porous material is connected with a suction port 158 which is connected to an i-pump. Above the porous material there are sensors 160 which themselves are connected to the i-pump. Above that there is a needle plate 162 which is connected to the inside of the housing through an elastic diaphragm 164.

There are spring-like devices 166 which space the needle plate 162 from the top plate 168 of the housing. One or more ventilation ports 170 are provided to the housing. A drug port 172 is provided for the delivery of drugs through the needle port 152 as necessary. The drug port is also connected to the i-pump, as are the sensors 160. The device operates with a programmable i-pump which activates suction in the suction port. The suction passes through porous material and draws tissue or skin into the tissue port. The suction draws the needle plate downwardly and then the needle enters the tissue and the needle plate activates the sensor. The sensor relates to the i-pump the condition in which drugs can be delivered. Suction is then stopped. The elastic diaphragm and the elastic elements retract the needle. A MEMS incorporated into the remote device may deliver vacuum to stabilize the skin and a MEMS may deliver the needle plate.

As shown in FIG. 12 there is a feature where the needle plate may be part of an array to deliver several needles and having sensors into tissue. As such, several needles 26 can be arranged with sensors 200 at the proximal end of the needle. The micro-needles are connected with the needle plate 202. The sensor in some cases may be the needle itself or may be incorporated into the needle. The sensors can measure blood glucose and an i-pump can deliver the appropriate insulin through the needle.

A system with porous material is shown in FIG. 13. The porous material may incorporate anti-microbial agents either on the surface or impregnated to gradually leach out during the life of the device. Suction might activate a leaching process to sterilize tissue. The second port in the porous material can provide for drug irrigation or an anti-microbial agent prior to or activation of the device. The two suction ports, as illustrated in FIG. 13 are namely the tissue suction port 204 and the drug port 206 which may be coextensive or separate as required. The tissue suction port is for causing the tissue to be drawn into the device so it can be stabilized when the needle 26 interacts with the tissue. The needle suction facilitates movement of the needle and/or irrigation of the tissue. Multiple suction ports may be added as required.

As shown in FIG. 14 there is an arrangement where the needle 26 is configured essentially to enter the tissue substantially at right angles. The needle 26 is right-angularly connected with the needle plate 208. As shown in FIG. 15 the needle 26 is constructed to enter the tissue obliquely, for instance at 45°. The needle 26 is located with the needle plate 210 at about 45°. Various angulations as such may be fashioned as required.

As shown in FIG. 16, an array of micro-needles 26 which may pass through different portions of the cutaneous interface 38 at different sites. The operation of each of the needles 26 can be electromagnetically controlled through MEMS 212 mounted on, with, or in relation to each needle plate 214. An electromagnetic element 216 may be mounted in adjacency with the magnets 212 to effect operation. A suitable sensor 218 is mounted towards the tip of the needle. Any suitable releasable mechanisms such as an electric switch or micro-switches may be applicable.

During the suction process the MEMS units 216 are operative and can hold or repel the needle plates or arrays, and four are illustrated in FIG. 16 as appropriate. Only one needle may penetrate the tissue as required. A fourth needle as indicated can use the sensor. The advantage of such smart arrays is their selective locations of penetration are possible. Multiple drug deliveries can be achieved. A sensor 218 can be configured to regulate delivery. The array can operate with a MEMS controlled by a microprocessor or i-pump.

A self-administering system with the device and method of the disclosure is particularly advantageous. This could be for emergency use, for instance for administering a shot for something like anthrax vaccine. A patient who is self-administering a drug or the like could be nervous and the skin could be shaking which would otherwise cause problems. The vacuum or suction stabilizes the tissue and this stabilizes the device relative to the tissue to prevent any sideways movement of the micro-needle array, which may otherwise damage the skin or break the needles. It also assists in achieving a consistent depth of needle penetration to avoid damage to other body structures such as tendons, nerves, and bones, while delivering consistent location of medical therapy.

The device can be used by a surgeon or other medical professional on internal body structures as well, rather than just the surface skin of an individual or other animal.

In some prior art devices, compressed air is used to deliver medication through the skin. Suction is better than compressed air for this purpose in that the suction stabilizes the skin and the device. Also, for a single use device, suction is preferable because compressed air could cause the device to expand.

The structure that contains the tubes that communicate the suction from the suction port to the various chambers can be a solid porous structure similar to the porous lava rock that is seen in fish tanks to create bubbles of air.

In another form, the device is constructed to be usable repetitively, such as for the injection of botox into a wrinkle. This can also involve the use of a drug metering system.

In other versions, there is puncturing of discrete portions of the membranes in the device that needs to work repetitively. If necessary, a system is provided for effective re-sealing of the punctures or a valve operated by a cam or electric switch may be used. In yet other systems, differentials in pressure, ideally the application of suction or a vacuum, can be applied to one side and then an opposite side to move a bladder or membrane in opposite directions as necessary. For instance, suction is applied to one side, and then suction is applied to the other side to move the needle back and forth.

In some embodiments, at least the tip or lead area of the housing 22 is relatively clear or transparent material so that the physician can see the area of skin to be punctured by the micro-needle array. This would have application, for instance, in the botox treatment where the doctor wants to follow a wrinkle line.

The diameter of the device can be made very small, just slightly larger than the micro-needle array in the interior. The needle 26 is not necessarily located in the center of the device.

Different advantages of the disclosure include the characteristics of the ability to preload the device with a drug, vaccine, or the like. This minimizes time for administration. It also facilitates the correct amount of preloading of the material to be injected. The vacuum grasping of the skin to a portion of the device facilitates stability of the skin and tissue prior to and during the injection of the material. The needle 26 acts automatically to puncture the skin and penetration effected to the correct skin depth. The content of the device can be in cartridge form and delivered by vacuum or pressure. The operation can be a single one-action process. This one-action process can be effected in the sequence indicated. After automatically activating the device to apply suction to stabilize the skin, the subsequent steps of injection and retraction can take place automatically. After use the needle retracts into the device. This increases the safety of the system.

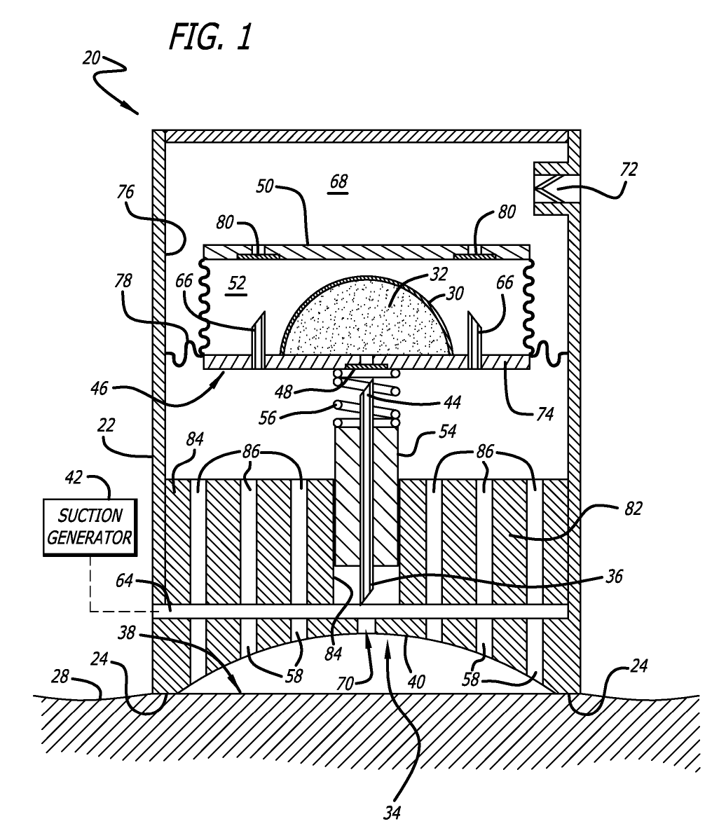

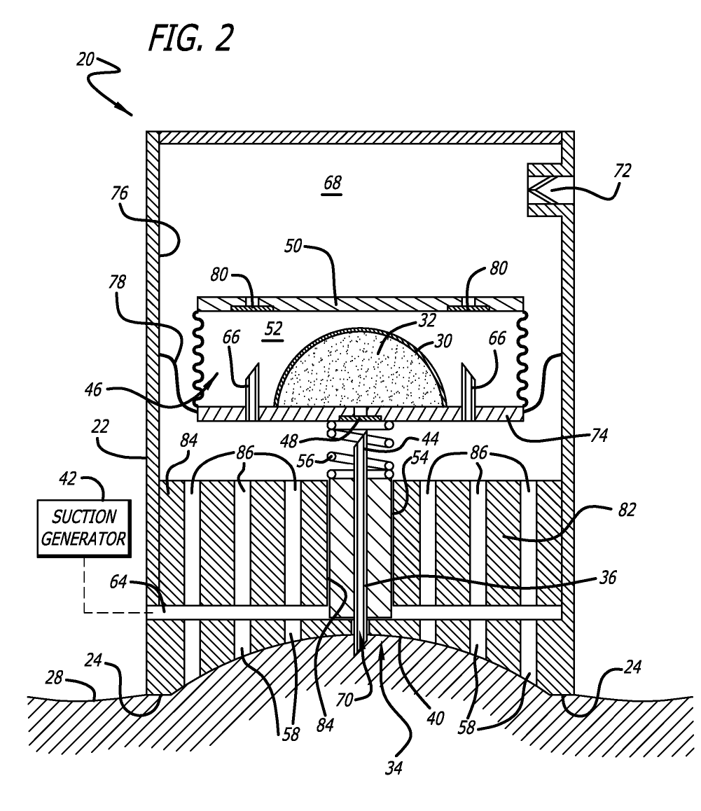

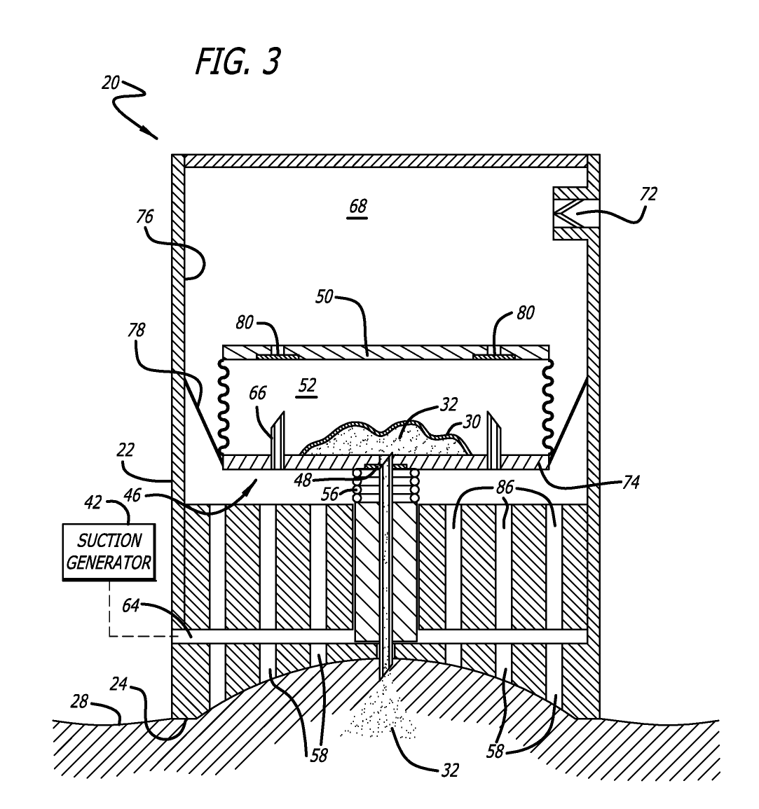

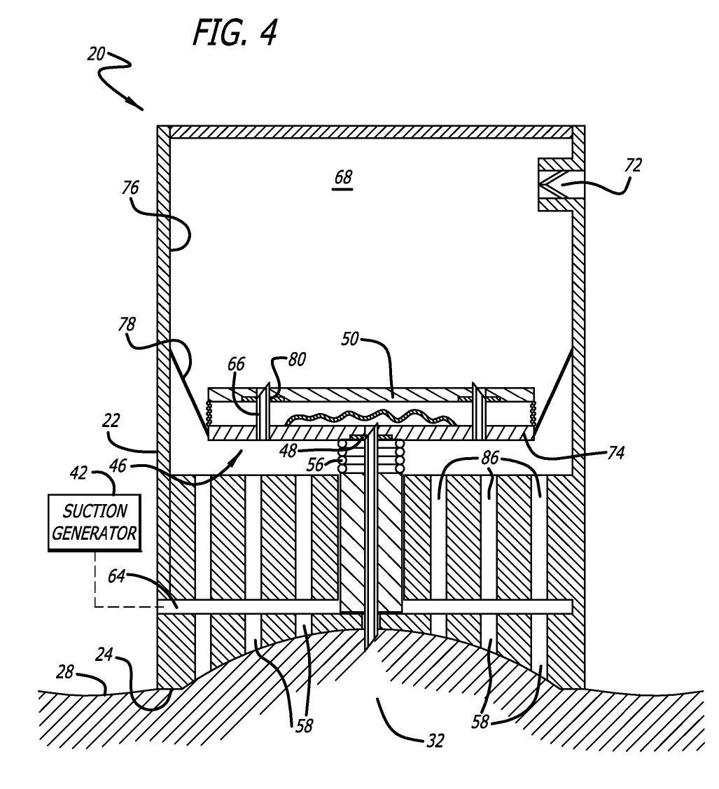

Other advantages include the multiple simultaneous drug delivery, multiple simultaneous needle punctures, the simplified ability to access difficult body sites, and the ability to use micro-needle arrays since the tissue is stabilized. The delivery action can be by vacuum, MEMS, sensor response, or micro-needles, for instance, when the cutaneous layer engages the surface 40 in FIG. 1. As such FIG. 1 can, for instance, be considered an example of a micro-needle approach to drug delivery. The micro-needle delivery of substance to the skin can be effected by many different techniques, including the spring loaded release when the cutaneous layer reaches certain positions or a vacuum or pressure level is sensed in the array area in relation to the cutaneous layer.

The overall system can be used similarly to the manner of grasping a pencil or pen, and different gripping mechanisms can be provided on the exterior of the body. There can also be one or more color indicators on the device to indicate the condition of the device. For instance, one color can be provided to indicate the device has not been used, a second color can be shown to indicate the device is penetrating the skin, and the third color can be used to indicate the device has been used and has been retracted and can now be discarded. These colors can show through one or more windows provided on the exterior of the body holding the device.

The device is essentially contained in a syringe-type barrel and contains multiple chambers, namely the suction chamber, ventilation chamber, drug-containing chamber, and tissue securing area.

Different mechanisms can be used to organize the exact sequence and operation of some the components of the device. For instance, although the spring mechanism is indicated in the preferred example to become operative only after delivery of the drug from the drug bladder, there may be systems where the spring does become operative slightly before or even after a delay of delivery of the drug. In this case the exact configuration of some of the components and application of some of the suctions and/or pressure in the device can vary for preferred applications.

The signaling system to indicate usage of the device can be a color indicator as well as an audible indicator. The audible indicator could operate as a whistle-type effect, by providing an aperture with a suitable reed-type valve which will emit a sound when the suction or pressure is applied to the aperture.

Other characteristics of the disclosure can include the provision of one or more adhesives or sticking elements to facilitate the adherence of the leading end of the device to the skin or tissue. Such an adhesive can be provided around the peripheral area of the device.

In other systems of the device there can be multiple tissue receiving ports with micro-needles located therein to provide a cell-type structure for the device. These tissue ports can be provided in a series of parallel locations in the device. The overall device cross-section can adopt any appropriate shape. As such, although the device may normally appear to be cylindrical when there is a single tissue port and retractable needle in a system. Where there are multiple tissue ports the overall device can have any other cross-sectional shape. The shape can, for instance, be square or elongated.

A common source for providing suction can be provided to each of the particular cells of the multiple systems. After use of a first cell the suction can be applied to a second and subsequent cell as required. Different cells may operate sequentially for suitable activation of a trigger by the doctor or the patient. In some other forms of the disclosure one or more additional biasing systems may be provided in appropriate places to facilitate the smooth and timely action of the components that such smooth action could accelerate the operation of some component or delay the action of some component as the case may be.

In yet other forms of the disclosure there can be a system whereby the needle delivers injectable material into tissue at multiple different delayed times. There can be a system where there is an automatic needle delivery system in which the needle penetrates the skin only during delivery of the drug. In different situations, small needles such as micro-needles can be used and this has the advantage of less pain for the patient. The easy removal and placement of the device is facilitated by the system. There can also be a situation where several needles can be incorporated where the one drug is delivered multiple times. There can be different sensors provided for each of the needles, and the needles can operate in sequence or simultaneously as required.

The pump for applying the injection can be programmable so that the micro-needles can be operated sequentially. This programming can be effected by electronic and/or mechanical means. As required, various degrees of complexity can be provided for most sophisticated systems for implementing the disclosure in its multiple uses and/or in arrays where needles and sensors are to be used. The sensor can be associated with a suction pump, bladder, or needle and there can be one or more measuring devices in the device, for sensing and measuring bodily conditions before, during, and after application of injectable material to the body.

One or more anti-microbial agents can be provided to the device on appropriate surfaces or impregnated so as to facilitate hygienic use and sterilization of components and/or the tissue prior, during, and after application of the device.

One or more areas of porous material can be provided to the device. For instance, one porous material may be provided around the tissue suction portion and a second porous material may be provided around the area relating to the needle suction. An impermeable region may be provided between those two porous materials. This can regulate the effect of the applied suction on the different components of the device.

In yet other forms of the disclosure, the needle can be directed in a substantially longitudinal direction with the overall longitudinal shape of the device there can be situations where the needle is orientated at an angle which is non-longitudinal relative to the device. There can also be situations where there are multiple needles arranged around the area which stabilizes the tissue, and each of these micro-needles can be directed at different angles relative to the device. We can penetrate the tissue at the appropriate angle with the tissue stabilized in the tissue port. One or more release mechanisms can be used with each of the respective needles. Such release mechanisms can be magnetic or electromagnetic. This may be a required operation of the electromagnetic systems, which can operate with a delay or in a regulated programmable fashion relative to the application of the suction process for securing the tissue in the tissue port.

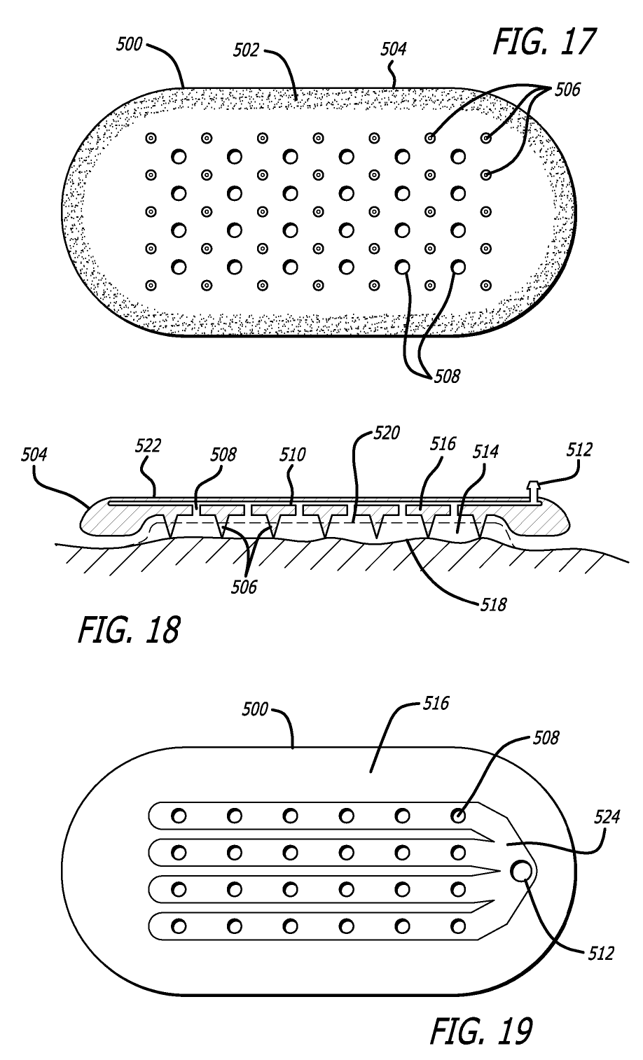

With further reference to the drawings, namely FIGS. 17 to 23 there is a device which permits effective engagement of micro-needles with a cutaneous layer thereby to permit for a substance to pass effectively to the cutaneous layer. There is a first substrate of flexible or non-flexible material having a transverse area and a peripheral edge. An array of micro-needles is arranged about the area for effectively engaging the cutaneous layer.

The transverse area within the peripheral edge of the material and with which the micro-needles connected are for receiving the surface of the cutaneous layer. The proximal ends of the micro-needles effect a passage of a substance with the micro-needles to the cutaneous layer.

There can be a bladder related to the first substrate for acting on the first substrate to cause proximal ends of the micro-needles to pass to the cutaneous layer for passage of a substance associated with the micro-needles to the cutaneous layer. This passage is usually into and below the cutaneous layer. This delivery of a substance can be by piezo electric effect, vacuum pump, micropump with an actuator, or MEMS delivery.

There is a connector for a suction generator for creating a suction force in the bladder thereby to urge the cutaneous layer towards the area within the peripheral edge of the material. The suction causes the micro-needles and cutaneous layer to move relatively closer to each other. This permits the proximal ends of the micro-needles to pierce the surface of the cutaneous layer. The passage of a substance from the micro-needles to the cutaneous layer can be by means of fluid passing through the micro-needles, when they are a format having a bore. Alternatively, if the micro-needles are solid, the substance can be layered on the surface and it is time release absorbed below or into the cutaneous layer.

The bladder includes apertures located about and spaced from the micro-needles. The suction is transmitted though the apertures to draw the cutaneous layer into engagement with a face of the substrate which is directed in the same direction as the proximate ends of the micro-needles.

The bladder communicates with a space between the needle array and the cutaneous layer.

A second substrate is substantially parallel to the first substrate and is part of the bladder formed with the first substrate and the peripheral edge. The bladder permits for the suction to be drawn in the area around the array of micro-needles.

In another form, the bladder is at least partly formed by a space between a first substrate and a closure structure above the layer. A port in one of the layers permits for connection to a suction generator. The port is connected to a suction array of apertures spaced about the micro-needle array. A differential pressure caused by the suction in the bladder effectively causes the micro-needles to move transversely between a position of repose withdrawn relative to the cutaneous layer towards a position relatively closer to the cutaneous layer.

The bladder is formed in part of elastomeric material and part of this is the first substrate. The array of micro-needles and the array of ports is arranged about the first substrate.

The surface of the substrate being the transverse area receives the cutaneous layer under action of the suction force. This stabilizes the cutaneous layer prior to and during piercing of the cutaneous layer by the proximal end of the micro-needles.

As this is a micro-needle array, there are multiple micro-needles in relative adjacency with each other. This creates multiple piercings of the cutaneous layer.

The micro-needles are mounted with a movable first substrate, so that in one form the first substrate includes a surface with concavities and the micro-needles are mounted in the concavities, and the apertures are located in the concavities. In another form the surface is substantially flat and the micro-needles and apertures are located to extend from the flat surface.

Instead of a full bladder or a form of the bladder, there can be considered to be a passageway for transmitting suction pressure on the cutaneous layer. The passageway is located in the space between the micro-needle array. The passageway communicates with the apertures between the micro-needles for transmitting the suction. The apertures are located about the micro-needles.

The passageway permits for suction to be drawn in the area around the array of micro-needles thereby causing the micro-needles and cutaneous layer to be drawn together in relatively closer contact.

As indicated, in some cases the micro-needles are hollow and fluid passes through the micro-needles to the cutaneous layer. In other cases, the micro-needles are solid and fluid passes from the surface of the micro-needles to the cutaneous layer.

In use, there is a cavity is formed between the first substrate and a cutaneous layer. The suction drawn into the cavity around the array of micro-needles causes the micro-needles and cutaneous layer to be drawn together in relatively closer contact.

There can be a third layer spaced relative to the second substrate layer at least partly forming a chamber between the second layer and third layer. There can be a biasing device for urging the first substrate from the cutaneous layer. An applied pressure can be used to counteract the bias and cause the micro-needles to engage and enter the cutaneous layer.

As such there is a mounting for supporting micro-needles with the first layer. The differential pressure causes the micro-needles to move transversely between a position of repose as caused by the spring. The movement is towards the cutaneous layer.

In yet another form there is selectively a spring or electromagnetic system for causing the micro-needles to move relatively with the cutaneous layer. The spring or electromagnetic system acts to urge the cutaneous layer towards the area within the peripheral edge of the material. This is a stabilizing force to the cutaneous layer. The micro-needles and cutaneous layer are caused to move relatively closer to each other and thereby permit the proximal ends of the micro-needles to pierce the surface of the cutaneous layer. Fluid can be expelled into the proximal end of the needle and thereby permit the expulsion of fluid into the cutaneous layer.

The needle array are micro-needles measuring about 25 to about 300 microns in height, are selectively biodegradable, and selectively made of silicon and formed using a MEMS process, and are formed with a density of hundreds of micro-needles in an area of about one square centimeter.

As shown in FIG. 17 the flexible patch 500 can be formed of a suitable polyurethane or polyurethane plasticized material which is flexible, and which can selectively be a typical pressure adhesive based on pressure sensitive tape material. The patch 500 is formed as part of a first substrate 516. An adhesive 502 is arranged around the perimeter of edge 504 of the material. Internally arranged and transversely within the perimeter 504 there is an array of the micro-needles 506. These can be hollow or solid depending on the particular substance, drug, or the like to be administered to a patient.

Arranged in an array between the micro-needles 506, there is an array of apertures 508. These apertures go through the plane and surface of the material 500 and extend to the top of the patch 500 above the array of micro-needles 506. The micro-needles 506 are below the patch 500. The apertures enter a space or vacuum bladder 510 which is connected to a port 512 thereby to permit the creation of a vacuum through the bladder 510. This causes a suction effect in the space 514 which space 514 is formed by the rear face of the substrate 516 and a cutaneous layer 518. This vacuum causes the cutaneous layer 518 to be lifted as indicated by the dotted line 520.

The bladder is formed by the first substrate 516 and a substrate 522. The top of the first substrate 516 can have a different format and define a passageway 524 which connects with the apertures 508.

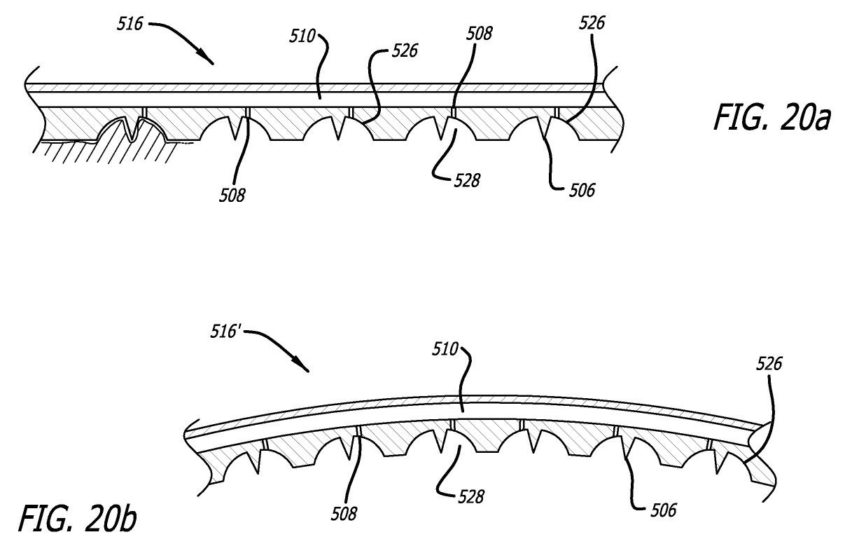

As shown in FIG. 20A there is a substrate configuration 516 with concaved sections 526. The micro-needles 506 extend from the concave sections 526. The ports 508 extend through the substrate 516 between the concavity sections 526 into the area 510 which would be about the substrate 516. The apertures 508 are set in the concavities 526 adjacent to the needles 506. This is an example of the application of suction to cause the cutaneous layer to adopt a different shape of engagement with the underside of the layer and then with the underside of each concave cell housing the micro-needle.

FIG. 20B is a different form of configuration where the application of a suction force causes the cutaneous layer to adopt two different forms of curvature or concavity. One form is across the general interface of the array, and the other form is with each cell housing the micro-needle itself.

In use, the flexible micro-needle array system operates generally as follows. The flexible array is placed on the skin with adhesive 502 adhering to the skin. A vacuum is applied to the suction or vacuum source port through the vacuum pump acting on the vacuum array. The micro-needle array enters or engages positively with the skin when the cutaneous layer 518 is drawn upwardly under the affect of the suction.

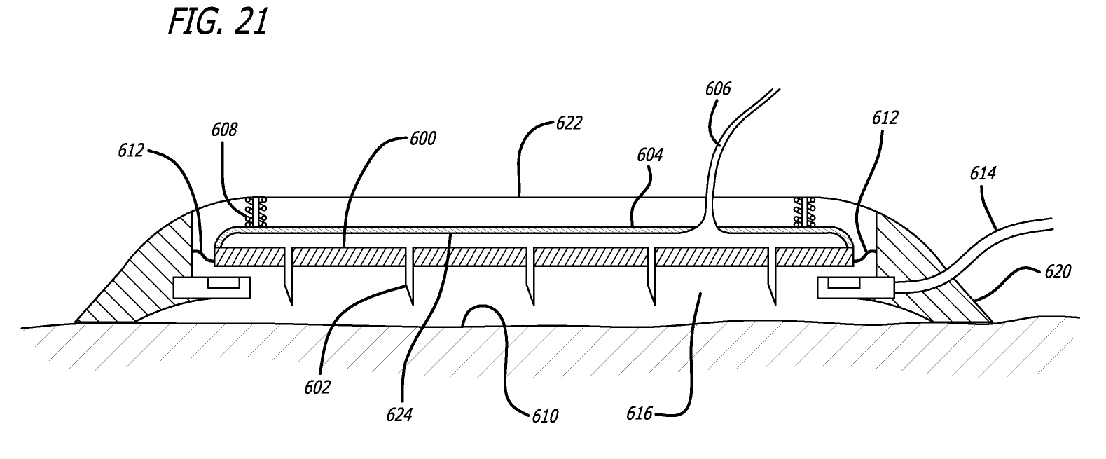

In the configuration shown in FIG. 21 there is a vacuum delivery system with a non-flexible patch and micro-needle array. The first substrate 600 supports micro-needles 602. These micro-needles are hollowed so that a fluid substance can be delivered through them. The substance is directed to the bladder 604 from the delivery tube 606.

Biasing means 608 acts to suspend the substrate 600 above the cutaneous layer 610. Additional biasing and spring means 612 are on the sides of the substrate 600 thereby facilitating suspension of the substrate 600. A vacuum from a suction port 612 is provided around and towards the edge of the patch 620. An outlet port 614 is provided to draw the vacuum from the space 616 and thereby cause the cutaneous layer 610 to rise relatively towards the micro-needles 602. The overall housing 622 is provided above the substrate layer 624 which is a second substrate layer and forms part of the chamber through which fluid can be introduced into the needles 602.

This configuration uses a vacuum, spring or electromagnetic means to stabilize the substrate 600.

An advantage of the system is that this can prevent force on the micro-needles which can cause hemorrhaging or breakage or skin tearing. The micro-needles can be used for complete drug delivery or for delivery of DNA, large molecules, proteins, etc. The system provides for controlling the depth of the micro-needles and this depth control minimizes pain.

In the configuration in FIGS. 20A and 20B, the micro-needles may be relatively longer than the micro-needles used in configuration of FIGS. 17, 18, and 19. The vacuum array may be removed after the micro-needles deliver the substance to the cutaneous layer. Similarly, these micro-needles can be used to deliver substances to internal organisms in different situations.

The delivery of a substance can be through holes in the needles or pressure by diffusion, osmotically, or by ultrasound. As such the delivery can be actively or passively effected.

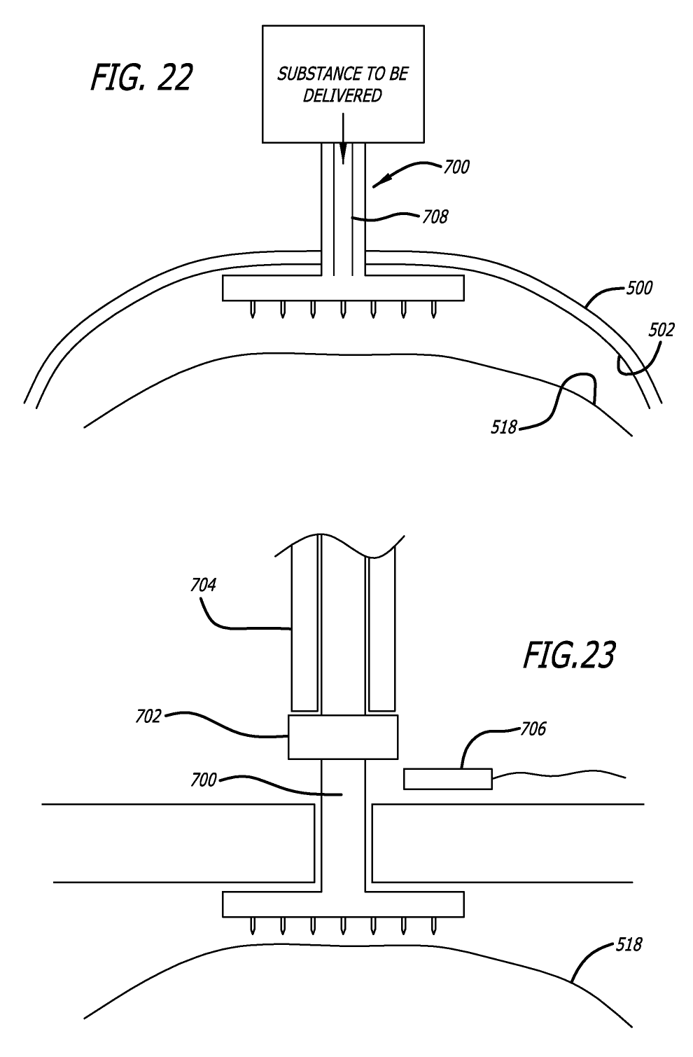

In the configuration of FIG. 22, there is shown figuratively the micro array related to a cutaneous layer. The adhesive material of a flexible patch extends to the outer side of the needle array. The delivery means for the substance to the micro-needles is shown above the micro array patch. A post 700 supports the array. A substance to be delivered is shown in relation to the post and can be delivered through a bore 708.

In FIG. 23, the micro array of micro-needles is supported on a post 700. There is a step 702 which operates with a release mechanism 704 and sensor 706 so that the positioning of the micro array and the control to and away from the skin in different configurations is controlled.

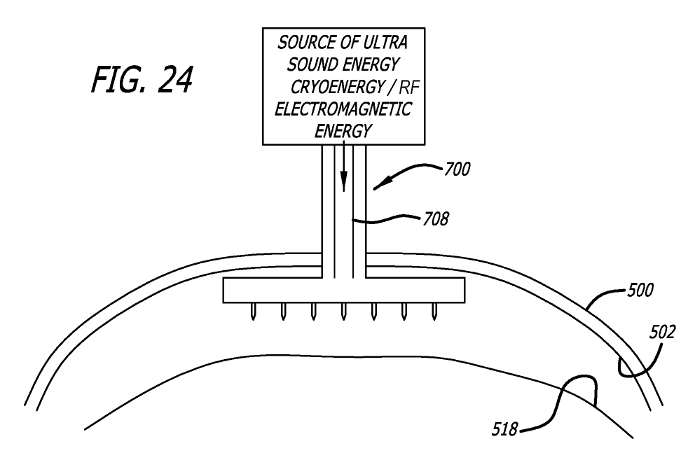

In FIG. 24 there is a system similar to FIG. 22. The post 700 is for conveying a supply of energy from an energy form 710 to the array. The energy can be cryoenergy, ultrasound energy, RF energy, or other electromagnetic energy. In different cases, different micro-needles can have different electrical polarities, uni-polar or bipolar.

In some applications, the energy or substance delivery or increased porosity of the cutaneous layer permits for the inflammation of the cutaneous layer and hence increased collagen production, and in different applications ablation of tissue, muscle or cells can be effected.

In yet other forms the micro-needles can interact with the cutaneous layer to enhance the porosity or permeability of the layer thereby permitting the enhanced absorption of substances, for instance creams spread on the outer surface of the cutaneous layer.

The micro-needles of the system can be formed with any appropriate material. Silicon processing can be used. In some cases tungsten or other bio-compatible micro-needles are appropriate. The micro-needles should not be bio reactive with the biology of the body. The micro-needles can be used for short-term or long-term use to deliver drugs or other substances to the body. Any pattern of micro-needles can be used. The system provides for the careful and controlled flow of drug or substances to the body using micro-needles with bio-compatible materials. The micro-needles can be molded with internal support posts as necessary to stabilize the needles. Putting appropriate polymers on the outside of the micro-needles secures the needle parts in case the micro-needles would otherwise be prone to breakage.

Although the micro-needles have been described with reference to injection on a cutaneous layer, it is possible to consider the use of the micro-needles on internal organs after appropriate entry within the body. It may be necessary to permit for suitable entry and exit to the body to provide for the appropriate delivery of drugs, substances, medications, ablation as appropriate. Applying electromagnetic energy through needles or micro needles can ablate tissue. The depth may be determined by the length of needle. Fluid such as saline may be applied through the needle to decrease the impedance.

The micro-needles themselves can be part of the patch of flexible material to contour to organs such as heart or liver where the part of the flexible material is made by techniques such as MEMS.

Although the disclosure relates in its examples to creating suction or a vacuum with the cutaneous layer, there could be situations where the application of pressure on the cutaneous layer is sufficient to stabilize the layer and thereby permit the effective interaction of the needles or micro-needles with the cutaneous layer.

Thus, in an alternative configuration, it could be possible that the first substrate does not have the apertures 508 or a vacuum part. It could be possible to apply a pressure so that the micro-needles move downwardly towards the cutaneous layer 518 and penetrate the layer. This would be effected by increasing the size of the bladder, placing a one way valve and using a passive vacuum as in a plunger

While the specification describes particular embodiments of the present disclosure, those of ordinary skill can devise variations of the present disclosure without departing from the inventive concept. The scope of the disclosure is to be determined by the following claims.

1. A device for permitting effective engagement of needles within a living body with internal body tissue thereby to permit for energy to pass effectively to the tissue comprising:
a) a first substrate of flexible material having a transverse area and a peripheral edge;
b) an array of needles arranged about the area for effectively engaging the cutaneous layer;
c) a bladder related to the substrate for acting on the substrate for causing proximal ends of the needles to pass to the cutaneous layer for passage of energy associated with the needles to the tissue;
d) the area transversely within peripheral edge of the material and with which the needles are connected being for receiving the surface of the tissue with which the proximal ends of the needles are to pierce to effect a passage of a energy with the needles to the tissue; and
e) a connector for a suction generator for creating a suction force in the bladder thereby to urge the tissue towards the area within the peripheral edge of the material;
the arrangement being such that suction causes the needles and cutaneous layer to move relatively closer to each other and thereby permit the proximal ends of the needles to pierce the surface of the tissue, and permit the passage of a energy from the needles to the tissue.

2. A device as claimed in claim 1 wherein the bladder includes apertures located about and spaced from the needles such that a suction transmitted though the apertures thereby draws the tissue into engagement with a face of the substrate directed in the same direction as the proximate ends of the needles.

3. A device as claimed in claim 1 wherein the bladder communicates with spaces between the needle array, and wherein there are apertures between the needles for transmitting the suction, and including a second substrate substantially parallel to the first substrate and the bladder being formed between the substrates and the peripheral edge.

4. A device as claimed in claim 1 wherein the bladder permits for suction to be drawn in the area around the array of micro needles thereby causing the needles and tissue to be drawn together in relatively closer contact.

5. A device as claimed in claim 1 wherein the bladder is at least partly formed by a space between a first substrate and a closure structure above the tissue.

6. A device as claimed in claim 1 wherein the bladder is at least partly formed by two spaced layers, and including a port in one of the layers for connection to a suction generator, the port being connected to a suction array about the needle array.

7. A device as claimed in claim 1 including a mounting for supporting needles, and wherein a differential pressure in the bladder effectively causes the micro needles to move transversely between a position of repose withdrawn relative to the tissue towards a position relatively closer to the tissue.

8. A device as claimed in claim 1 wherein the bladder is formed in part of elastomeric material, and wherein there is an array of needles and array of ports arranged about the first substrate, the array of needles and the array of ports being separated from each other.

9. A device as claimed in claim 1 including a surface on the transverse area, the surface being for receiving the tissue under action of the suction force, and thereby stabilizing the tissue prior to and during piercing of the cutaneous layer by the proximal end of the needles.

10. A device as claimed in claim 1 wherein there are multiple needles in relative adjacency with each other thereby to permit multiple piercings of the tissue.

11. A device as claimed in claim 1 wherein the needles are mounted with a movable first substrate.

12. A device as claimed in claim 1 wherein the first substrate includes a surface with concavities and wherein the needles are mounted in the concavities, and the apertures being located in the concavities.

13. A device for permitting effective engagement of needles with internal body tissue with a living body thereby to permit for a energy to pass effectively to the tissue comprising:
a) a first substrate of material having a transverse area and a peripheral edge;
b) an array of needles arranged about the area for effectively engaging the tissue;
c) a passageway for transmitting suction pressure on the tissue, to cause proximal ends of the needles to pass to the tissue for passage of a energy associated with the needles to the tissue;
d) the area transversely within peripheral edge of the material and with which the needles being for receiving the surface of the tissue with which the proximal ends of the needles are to pierce to effect a passage of a energy with the micro needles to the cutaneous layer; and
e) a connector for a suction generator for creating a suction force thereby to urge the tissue towards the area within the peripheral edge of the material;
the arrangement being such that suction causes the needles and tissue to move relatively closer to each other and thereby permit the proximal ends of the needles to pierce the surface of the tissue and permit the passage of energy from the needles to the tissue.

14. A device as claimed in claim 13 including apertures located about the needles such that suction is transmitted though the apertures thereby to draw the tissue into engagement with a side of the substrate directed in the same direction as the needles.

15. A device as claimed in claim 13 wherein the passageway communicates with spaces between the needle array, and wherein there are apertures between the needles for transmitting suction.

16. A device as claimed in claim 13 wherein the passageway permits for suction to be drawn in the area around the array of needles thereby causing the needles and tissue to be drawn together in relatively closer contact.

17. A device as claimed in claim 13 including a second substrate of material formed with the first material and having a space between the materials thereby to form the passageway.

18. A device as claimed in claim 13 including a port in the layer for connection to a suction generator, the port being connected to a suction array about the needle array.

19. A device as claimed in claim 13 including a mounting for supporting needles, and wherein a differential pressure in the bladder effectively causes the needles to move transversely between a position of repose withdrawn relative to the tissue towards a position relatively closer to the tissue.

20. A device as claimed in claim 13 wherein the passage is formed in part of elastomeric material, and wherein there is an array of needles and array of ports arranged about the first substrate, the array of needles and array of ports being separated from each other.

21. A device as claimed in claim 13 including a surface on the transverse area, the surface being for receiving the tissue under action of the suction force, and thereby stabilizing the tissue prior to and during piercing of the tissue by the proximal end of the micro needles.

22. A device as claimed in claim 13 wherein there are multiple needles in relative adjacency with each other thereby to permit multiple piercings of the tissue.

23. A device as claimed in claim 13 wherein the first substrate includes a surface with concavities and wherein the needles are mounted in the concavities, and the apertures being located in the concavities.

24. A device as claimed in claim 13 wherein the needles are hollow and energy passes through the needles to the cutaneous layer.

25. A device as claimed in claim 13 wherein the needles are solid and energy passes from surface of the needles to the cutaneous layer.

26. A device for penetrating internal tissue into a living body to permit for energy to pass through the tissue comprising:
a) a first substrate of material having an area and a peripheral edge;
b) an array of needles arranged about the area for piercing the tissue, the micro needles having a proximal end and a distal end;
c) a bladder or drug cartridge for containing fluid for passing across the tissue for injection below the tissue;
d) an area transversely within peripheral edge of the material and through which the needles are directed, and the area being for receiving the surface of the tissue about which the needles are to pierce to effect a passage of energy; and
e) a connector for a suction generator for creating a suction force at the area thereby to urge the tissue towards the area within the peripheral edge of the material and thereby provide a stabilizing force to the tissue;
the arrangement being such that the needles and tissue are caused to move relatively closer to each other and thereby permit the proximal ends of the needles to pierce the surface of the tissue; and permitting the passage of energy into the distal end of the needle and thereby permit the expulsion of energy into the tissue.

27. A device as claimed in claim 26 wherein the bladder communicates with the needles such that energy is transmitted through an outlet in the needles, and the bladder delivers the energy by piezo actuator.

28. A device as claimed in claim 26 wherein, in use, a cavity is formed between the first substrate and a tissue, and whereby suction is drawn into the cavity around the array of needles thereby causing the needles and tissue to be drawn together in relatively closer contact.

29. A device as claimed in claim 26 including a material layer at least partly parallel to and spaced relative to the first substrate layer thereby forming the bladder at least partly between the spaced layers.

30. A device as claimed in claim 29 including a third layer spaced relative to the second substrate layer at least partly forming a chamber between the second layer and third layer.

31. A device as claimed in claim 29 including a biasing device for urging the first substrate from the tissue.

32. A device as claimed in claim 26 including a mounting for supporting needles with the first layer, and wherein a differential force causes the needles to move transversely between a position of repose relatively withdrawn towards the tissue.

33. A device as claimed in claim 26 wherein there are multiple needles in relative adjacency with each other thereby to permit multiple piercings of the tissue.

34. A device for penetrating internal tissue inside a living body to permit for energy to pass through the tissue comprising:
a) a first substrate of material having an area and a peripheral edge;
b) an array of needles arranged about the area for piercing the tissue, the needles having a proximal end and a distal end;
c) selectively a spring or electromagnetic system for causing the needles to move relatively with the tissue;
d) an area transversely within peripheral edge of the material and through which the needles are directed, and the area being for receiving the surface of the tissue about which the micro needles are to pierce to effect an injection of fluid; and
e) thereby to urge the tissue towards the area within the peripheral edge of the material and thereby provide a stabilizing force to the tissue; the arrangement being such that the needles and tissue are caused to move relatively closer to each other and thereby permit the proximal ends of the needles to pierce the surface of the tissue; and permitting the passage of energy into the distal end of the needle and thereby permit the expulsion of fluid into the tissue.

35. A device as claimed in claim 1 wherein the needle array are micro needles measuring about 25 to about 300 microns in height, are selectively biodegradable, and selectively made of silicon, and are formed with a density of about several hundred micro needles in an area of about one square centimeter.

36. A device as claimed in claim 13 wherein the needle array are micro needles measuring about 25 to about 300 microns in height, are selectively biodegradable, and selectively made of silicon, and are formed with a density of about several hundred micro needles in an area of about one square centimeter.

37. A device as claimed in claim 26 wherein the needle array are micro needles measuring about 25 to about 300 microns in height, are selectively biodegradable, and selectively made of silicon, and are formed with a density of about several hundred micro needles in an area of about one square centimeter.

38. A device as claimed in claim 34 wherein the needle array are micro needles measuring about 25 to about 300 microns in height, are selectively biodegradable, and selectively made of silicon, and are formed with a density of about several hundred micro needles in an area of about one square centimeter.

39. A device for permitting effective engagement of needles with internal tissue inside a living body thereby to permit for energy to pass effectively to the tissue comprising:
a) a first substrate of flexible material having a transverse area and a peripheral edge;
b) an array of needles arranged about the area for effectively engaging the tissue;
c) a bladder related to the substrate for acting on the substrate for causing proximal ends of the needles to pass to the tissue for passage of energy associated with the needles to the tissue;
d) the area transversely within peripheral edge of the material and with which the needles are connected being for receiving the surface of the tissue with which the proximal ends of the needles are to engage to effect a passage of energy with the needles to the tissue; and
e) a connector for a suction generator for creating a suction force in the bladder thereby to urge the tissue towards the area within the peripheral edge of the material;
the arrangement being such that suction causes the needles and tissue to move relatively closer to each other and thereby permit the proximal ends of the needles to engage the surface of the tissue, and permit the passage of energy from the needles to the cutaneous layer.

40. A device as claimed in claim 39 wherein the bladder includes apertures located about and spaced from the needles such that suction transmitted though the apertures thereby draws the tissue into engagement with a face of the substrate directed in the same direction as the proximate ends of the needles.

41. A device as claimed in claim 39 wherein the bladder communicates with spaces between the needle array, and wherein there are apertures between the needles for transmitting suction, and including a second substrate substantially parallel to the first substrate and the bladder being formed between the substrates and the peripheral edge.

42. A device as claimed in claim 39 wherein the bladder permits for suction to be drawn in the area around the array of micro needles thereby causing the needles and tissue to be drawn together in relatively closer contact.

43. A device as claimed in claim 39 wherein the bladder is at least partly formed by a space between a first substrate and a closure structure above the tissue.

44. A device as claimed in claim 39 wherein the bladder is at least partly formed by two spaced layers, and including a port in one of the layers for connection to a suction generator, the port being connected to a suction array about the needle array.

45. A device as claimed in claim 39 including a mounting for supporting needles, and wherein a differential pressure in the bladder effectively causes the micro needles to move transversely between a position of repose withdrawn relative to the cutaneous layer towards a position relatively closer to the tissue.

46. A device as claimed in claim 39 wherein the bladder is formed in part of elastomeric material, and wherein there is an array of needles and array of ports arranged about the first substrate, the array of needles and the array of ports being separated from each other.

47. A device as claimed in claim 39 including a surface on the transverse area, the surface being for receiving the tissue under action of the suction force, and thereby stabilizing the tissue prior to and during engagements of the tissue by the proximal end of the needles.

48. A device as claimed in claim 39 wherein there are multiple needles in relative adjacency with each other thereby to permit multiple engagements of the tissue.

49. A device for penetrating internal tissue in a living body to permit for interaction with the cutaneous layer comprising:
a) a first substrate of material having an area and a peripheral edge;
b) an array of needles arranged about the area for interacting with the tissue, the needles having a proximal end and a distal end;
c) selectively a spring or electromagnetic system for causing the needles to move relatively with the tissue;
d) an area transversely within peripheral edge of the material and through which the needles are directed, and the area being for receiving the surface of the tissue about which the micro needles are to interact; and
e) thereby to urge the cutaneous layer towards the area within the peripheral edge of the material and thereby provide a stabilizing force to the tissue; the arrangement being such that the needles and tissue are caused to move relatively closer to each other and thereby permit the proximal ends of the needles to interact with the surface of the tissue; and permitting the interaction of the distal end of the needle with the tissue.

50. A device as claimed in claim 39 including a source of energy, the energy being selectively one of cryoenergy, ultrasound energy, RF energy, or other electromagnetic energy.

51. A device as claimed in claim 39 including having different micro needles with different electrical polarities.

52. A device as claimed in claim 39 including an energy source for applying energy to attain ablation of cutaneous or subcutaneous material or cells of organs, and selectively having an irrigation supply for applying irrigation through the needle hole.

53. A device as claimed in claim 39 including an energy source for applying energy to attain ablation of cutaneous or subcutaneous material or cells of organs, and selectively having an irrigation supply for applying irrigation through the needle hole.

54. A device as claimed in claim 39 including having the micro needles interact with the tissue to enhance the porosity or permeability of the layer thereby permitting the enhanced absorption of substances.

55. A device as claimed in claim 1 including effecting delivery of a substance to attain increased porosity of the tissue permits for the inflammation of the cutaneous layer.

56. A device as claimed in claim 1 to effect an ablation of cutaneous or subcutaneous material or cells of organs.

57. A device as claimed in claim 1 including having the micro needles interact with the tissue to enhance the porosity or permeability of the tissue thereby permitting the enhanced absorption of energy.

58. A device as claimed in claim 49 including a source of energy, the energy being selectively one of cryoenergy, ultrasound energy, RF energy, or other electromagnetic energy.

59. A device as claimed in claim 49 including having different micro needles with different electrical polarities or selectively sensors or needles to effect removal of biologic substance.

60. A device as claimed in claim 49 including applying energy to attain increased porosity of the cutaneous layer permits for the inflammation of the cutaneous layer.

61. A device as claimed in claim 49 including an energy source for applying energy to attain ablation of subcutaneous material or cells of organs.

62. A device as claimed in claim 49 including having the micro needles interact with the tissue to enhance the porosity or permeability of the layer thereby permitting the enhanced absorption of energy.

63. A device as claimed in claim 1 including an energy source for applying the energy at a level for ablating endocardial or myocardial tissue.

64. A device as claimed in claim 13 including an energy source for applying the energy at a level for ablating endocardial or myocardial tissue.

65. A device as claimed in claim 26 including an energy source for applying the energy at a level for ablating endocardial or myocardial tissue.

66. A device as claimed in claim 34 including an energy source for applying the energy at a level for ablating endocardial or myocardial tissue.

67. A device as claimed in claim 39 including an energy source for applying the energy at a level for ablating endocardial or myocardial tissue.

67. A device as claimed in claim 49 including an energy source for applying the energy at a level for ablating endocardial or myocardial tissue.

68. A device as claimed in claim 1 including increasing the conductivity of the tissue through the needles, the conductivity being increased selectively by the irrigation fluid dispersed to the tissue.
